(19) United States
(12) Patent Application Publication
(10) Pub. No.: US 2024/0180755 A1
(43) Pub. Date: Jun. 6, 2024

(54) Sanitary Pads on a Roll

(71) Applicant: Egal Pads Inc., Somerville, MA (US)

(72) Inventor: Thomas E. Devlin, Winchester, MA (US)

(21) Appl. No.: 18/286,241

(22) PCT Filed: Nov. 19, 2021
(86) PCT No.: PCT/US2021/060029

§ 371 Date: Oct. 10, 2023

(51) Int. Cl. A61F 13/15; A61F 13/47

(52) U.S. Cl. CPC A61F 13/15707; A61F 13/4702

(57) ABSTRACT

A sanitary-pad roll includes a core and a strip that is wrapped around the core. The strip, when unrolled, extends along a longitudinal direction that is perpendicular to a transverse direction. In addition, the strip includes an impermeable layer, an absorbent layer, and a weakened region that extends along the transverse direction and defines segments that are detachable from the strip. These segments, when detached, form corresponding sanitary pads. The impermeable layer, which is wider than the absorbent layer, is folded over along the longitudinal direction to form an overlap, thereby enclosing the absorbent layer.

### FIELD OF THE INVENTION

The present invention relates to sanitary pads for public use.

### BACKGROUND

Those who use public toilets take it for granted that toilet paper will be available. However, those who menstruate encounter a different world. As a result, those who menstruate often to carry sanitary pads or tampons at all times in case their menstrual cycles begin unexpectedly.

In many cases, one discovers the need for a sanitary pad only while already using the toilet. If one has forgotten to bring the necessary supplies, an inconvenient and potentially embarrassing predicament arises

Some public restrooms have tampons and pads for sale in a vending machine. But for someone who is already sitting on the toilet, this is hardly convenient. One would still have to dress and exit the stall to retrieve the product. To make matters worse, one would have to find the proper change to use in the vending machine.

### SUMMARY

In one aspect, the invention features a strip of individually die-cut sanitary pads that has been rolled onto a core. Each sanitary pad has adhesive for attaching to an undergarment. The strip is wide enough to fold over the sanitary pad, thus preventing leakage and protecting it from contamination. Between each pair of sanitary pads is an inter-pad gap comprising backing material that has been perforated to facilitate separation of individual segments, each of which contains a sanitary pad.

In some embodiments, the backing material is welded to itself, thus creating a sealed compartment between each sanitary pad. This prevents a sanitary pad adjacent to the exposed end of the strip from becoming wet or contaminated.

Ideally, the roll is a cylindrical structure having a circular cross section in which the radius is independent of angle. A difficulty that can arise is the possibility that inter-pad gaps will align with each other. This causes the radius to be smaller at certain ranges of angle. As a result, the roll develops an eccentric cross section.

In an alternative embodiments, the sanitary pads are kept together on a continuous strip instead of being individually die cut. Each sanitary pad has an absorbing region that is enclosed by a seal. The seal is formed by welding a top layer of the strip to a bottom layer of the strip. Such welding is carried out by application of energy, whether it be thermal energy or acoustic energy. Such a seal prevents bleeding at edges of the strip.

In some embodiments, the seal does not completely surround the absorbing region, thereby permitting the absorbing region to remain a continuous strip rather than being interrupted at each sanitary pad. This speeds up the manufacturing process but at the cost of leaving an unsealed section that increases risk of leaking.

In one aspect, the invention features a sanitary-pad roll having a core and a strip that is wrapped around the core. The strip, when unrolled, extends along a longitudinal direction that is perpendicular to a transverse direction. In addition, the strip includes an impermeable layer, an absorbent layer, and a weakened region that extends along the transverse direction and defines segments that are detachable from the strip. These segments, when detached, form corresponding sanitary pads. The impermeable layer, which is wider than the absorbent layer, is folded over along the longitudinal direction to form an overlap, thereby enclosing the absorbent layer.

In some embodiments, the weakened region comprises perforations that extend through the strip.

In other embodiments, the core comprises a cardboard tube.

Embodiments include those in which the sanitary pad is die cut and those in which the sanitary pad comprises wings.

Among the embodiments are those in which the overlap is bonded. These include embodiments in which the overlap is bonded by heat and those in which it is bonded by mechanical staking.

Embodiments further include those in which the absorbent layer comprises a plurality of absorbent regions separated by inter-pad gaps, each of the absorbent regions corresponding to a sanitary pad. In such embodiments, the weakened region extends across the inter-pad gap.

Also among the embodiments are those in which each segment comprises a first end and second end that is separated from the first end along the longitudinal direction. In these embodiments, the first end comprises a first seal and the second end comprises a second seal. Between these seals is a length of strip that extends between them. The wherein the weakened region extends across this length.

In still other embodiments, the strip comprises a first segment comprising a first seal, a second segment comprising a second seal, and a length that extends between the first and second seals. This length has a thickness that is less than that of either the first and second seal. Meanwhile, the first and second seal each have a thickness that is less than that of the absorbent layer.

In still other embodiments, the strip comprises a length that separates adjacent segments, the length having thickness that is between half a millimeter and two millimeters.

In some embodiments, each segment comprises a first and second end, the second end being separated from the first end along the longitudinal direction. The strip comprises a seal that extends from the first end to the second end and the weakened region extends across the seal.

In other embodiments, the strip comprises a seal that separates adjacent segments and that has a thickness that is less than that of the absorbent layer.

Embodiments include those in which the strip has a thickness that varies periodically along its length at a spatial frequency that is a function of the number of sanitary pads on the strip and the length and those in which the strip's thickness has a first value along a sanitary pad and a second value between sanitary pads, thereby defining a square wave. Among these are embodiments in which the second value transitions between third and fourth values, thereby defining another square wave.

Also among the embodiments are those in which the strip has a thickness that varies along the longitudinal direction, the thickness having a first value along a first section, a second value along a second section, a third value along a third section, the second value along a fourth section, and the first value along a fifth section. The first and fifth sections each comprise a sanitary pad. The second, third, and fourth sections are between the first and fifth sections and the third section is between the second and fourth section. In such embodiments, the the third value is less than the second value and the second value is less than the first value.

The embodiments further comprise those having pairs of notches in the absorbent strip. These notches extend along the transverse direction towards each other and mark boundaries between sanitary pads and those having center-punched holes through the strip at intervals that define an extent of a longitudinal extent of a sanitary pad.

Also among the embodiments are those in which the strip comprises a distal segment that is attached to only one other segment. This distal segment comprises a distal edge that extends along the transverse direction. In some of these embodiments, the distal edge comprises sealed corners on either side of an unsealed space. In others, the distal edge comprises a sealed center section and unsealed corners.

Also among the embodiments are those in which the impermeable layer comprises a backing material that is welded to itself to form the overlap, thus creating a sealed compartment for each sanitary pad to prevent the sanitary pad from becoming wet or contaminated.

In some embodiments, the sanitary pad is not die cut and is instead formed by tearing across the strip at the weakened region. In others, for example where the sanitary pad has been die cut, the sanitary pad is released after tearing across the strip at the weakened region.

Embodiments further include those in which the absorbent layer is discontinuous along the strip's longitudinal direction and those in which the absorbent layer is continuous along that direction. Among the latter are embodiments in which the absorbent layer comprises features disposed periodically along the layer at intervals equal to a length of the segment. Examples of such features include notches in the absorbent layer and holes in the absorbent layer. In other embodiments, the manufacture includes a wall-mounted dispenser for placement adjacent to a toilet. Such a dispenser includes a spindle. The core is placed over the spindle such that pulling on the strip causes a torque that rotates said core. Among these are embodiments in which the spindle is one of two spindles, with the other spindle supporting a roll of toilet paper.

In another aspect, the invention features a segment that includes a sanitary pad disposed between first and second layers. The sanitary pad includes a backing layer, an absorbent fabric, an absorbent region between the backing layer and the absorbent fabric, and an adhesive layer disposed on the backing layer. The segment includes end margins at ends thereof and side margins along sides thereof. The first and second layers come together at these margins to form a seal around the sanitary pad. At least one of the end margins has been torn off to separate the segment from another similar segment. Removal of the first and second layers thus yields a sanitary pad.

These and other aspects of the invention will be apparent from the following detailed description and the accompanying claims, in which:

### DESCRIPTION OF DRAWINGS

FIG. 1 shows a dual-roll dispenser that dispenses toilet paper and sanitary pads;

FIG. 2 is a representation of a sanitary pad stored on the dispenser of FIG. 1 showing an absorbing region, and a sealing region that surrounds the absorbing region to keep fluid from leaking;

FIG. 2A is a variation of the sanitary pad shown in FIG. 2 but with wings that wrap around an undergarment for more secure placement;

FIG. 2B is a cross section of the sanitary pad of FIG. 2 with cross-sectional dimensions having been exaggerated to promote clarity of illustration;

FIG. 3 shows a roll of continuous of sanitary pads with an outside margin of the pad and the outer layer having been folded over on both sides to completely cover the pad;

FIG. 3A is a cross section of a sanitary pad from the roll shown in FIG. 3;

FIG. 4 shows various states associated with the assembly of a continuous strip of sanitary pads as shown in FIG. 1;

FIG. 4A is a segment of the continuous strip in FIG. 4 that has been separated showing the perforated edges;

FIG. 4B is a cross section of the continuous strip of FIG. 4 showing a long and substantially thin section between sanitary pad segments;

FIG. 5 is a representation of a continuous strip assembly with a cross-section as shown in FIG. 4B showing the offset that arises when thin sections align with each other in a roll;

FIG. 6 shows a strip of sanitary pads similar to that shown in FIG. 3 but without having been die cut and instead comprising a series of absorbing regions surrounded by sealing regions on a continuous strip;

FIG. 6A is as segment of the continuous strip assembly of FIG. 4 that has been separated showing its perforated edges;

FIG. 6B is a pad that has been removed from the assembly segment of FIG. 6A showing perforated edges.

FIG. 6C is a cross section of the continuous strip of FIG. 6 showing a shortened thin section;

FIG. 7 is a variation of the pad design of FIG. 6 in which the absorbing region and the sealing region are continuous;

FIG. 7A is a resulting segment of the pad design of FIG. 7 after being separated, showing a region that could leak;

FIG. 7B is a second variation of the pad design of FIG. 6 where the absorbing region and sealing region are continuous; and

FIG. 7C is a resulting segment of the pad design of FIG. 7B after being separated, showing corners that could leak.

### DETAILED DESCRIPTION

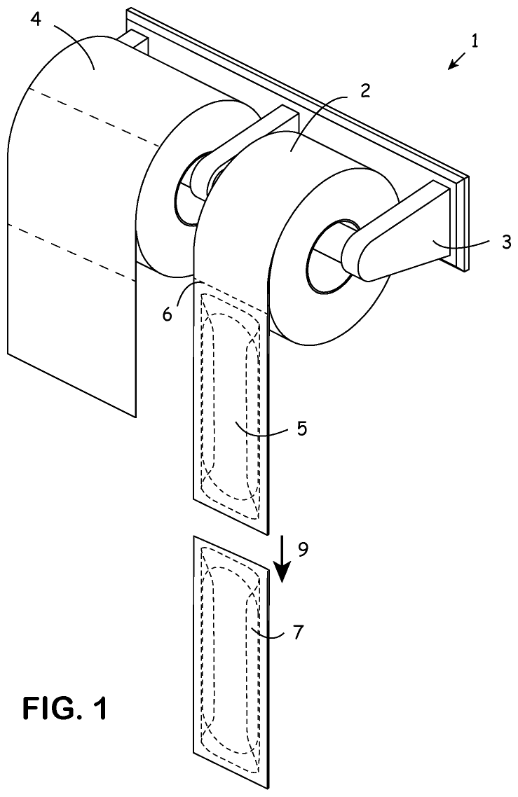

FIG. 1 shows a wall-mounted dual dispenser 1 having two spindles 3, one of which holds a toilet-paper roll 4 and the other of which holds a sanitary-pad roll 2. The dual dispenser 1 is typically mounted on a wall near a toilet.

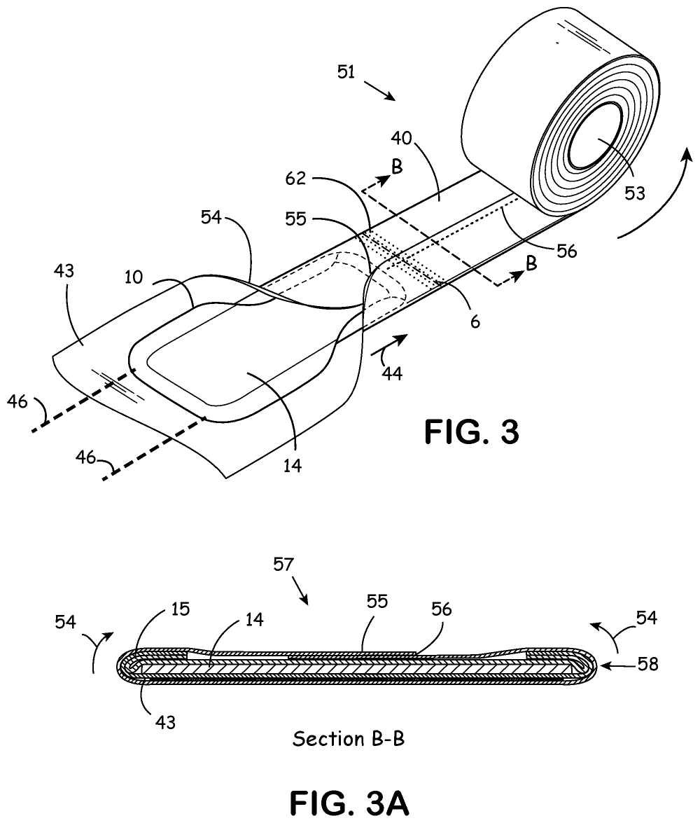

The sanitary-pad roll 2 comprises a strip 40, best seen in FIG. 3. The strip 40 comprises attached segments 5 that are attached to each other at a weakened section that has been weakened by perforations 6. Among these attached segments 5 is a distalmost attached segment 5 that is characterized by being attached to only one other attached segment 5.

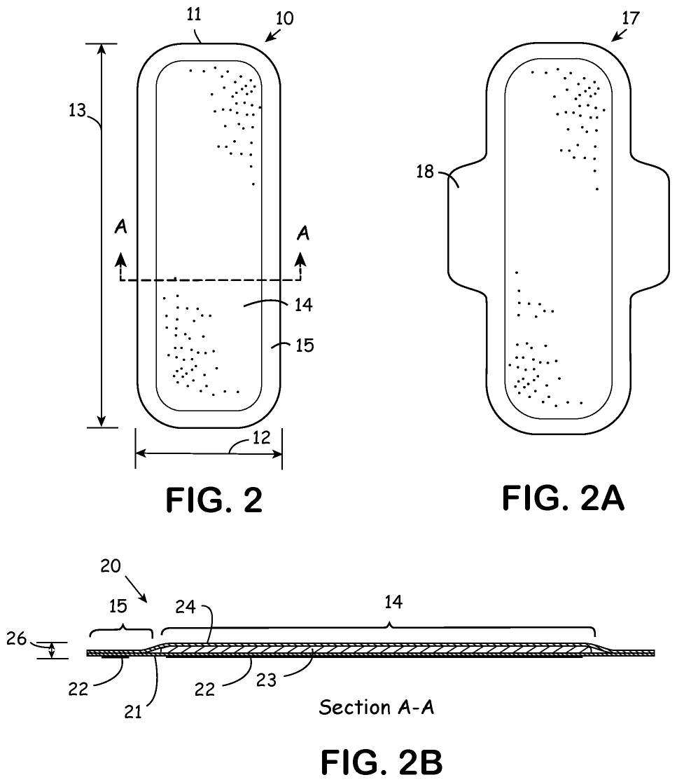

Referring now to FIG. 2, each attached segment 5 has a sanitary pad 10 within it. A user who requires a sanitary pad grasps the distalmost attached segment 5 and pulls along a tearing direction 9. This causes the perforations 6 to give way. As a result, the user transforms the attached segment 5 into a detached segment 7. The user then retrieves the sanitary pad 10 from the detached segment 7 so that it can be used for its intended purpose.

Referring now to FIG. 2, the sanitary pad 10 has a width 12 and a length 13. In typical embodiments, the sanitary pad 10 extends along its width 12 by between thirty millimeters and sixty millimeters and along its length 13 by between 140 millimeters and 240 millimeters. The sanitary pad's overall footprint 11 is determined by whatever die that is used to stamp out the pad 10. In some embodiments, such as that shown in FIG. 2A, the die used to form the sanitary pad 10 results in a profile 11 having laterally-extending wings 18 that wrap around an undergarment for added retention.

The sanitary pad 10 includes an absorbent region 14 surrounded by a seal 15. The absorbent region 14 absorbs fluids. The seal 15 prevents leakage of these absorbed fluids out of the absorbent region 14.

FIG. 2B shows a cross-section of the sanitary pad 10 of FIG. 2 along the line “A-A.” As is apparent from FIG. 2B, the absorbent region 14 comprises an absorbent material 23 that lies between a backing layer 21 and an absorbent fabric 24. An adhesive layer 22 permits the sanitary pad 10 to adhere to an undergarment when in use.

The absorbent fabric 24 extends beyond the perimeter of the absorbent region 14. As a result, a portion of the absorbent fabric 24 can be welded to the backing layer 21. This creates a fluid-impermeable seal 15. The overall thickness 26 of the seal 15 is between about half a millimeter and two millimeters.

In some embodiments, the absorbent material 23 comprises a low-density polyethylene film and the absorbent fabric 24 is a non-woven cotton fabric. Other embodiments include those in which the absorbent material 23 is cotton, cellulose, a laminate, or an assembly comprising a super-absorbent polymer.

An embodiment that relies on a super-absorbent polymer is particularly useful because of the super-absorbent polymer's ability to absorb considerable amounts of liquid. The super-absorbent polymer is sprayed on as a powder in a continuous operation. This results in a sanitary pad 10 that absorbs as much fluid as a thicker pad that relies on cotton or cellulose. The number of sanitary pads 10 that can be accommodated on a sanitary-pad roll 2 having a fixed maximum diameter is limited in part by the thickness of the sanitary pad 10. As a result, having sanitary pads 10 that are as thin as possible makes it practical to have enough pads on a roll to be cost effective.

FIG. 3 shows the continuous strip 40 of sanitary pads 10 rolled onto a core 53 that forms the sanitary-pad roll 2 shown in FIG. 1. The core 53 of the sanitary-pad roll 2 is preferably a cardboard tube much like the core of a typical toilet-paper roll 4.

The sanitary pads 10 shown in FIG. 3 have been placed along the continuous strip 40 sequentially in a rolling direction 44 on the strip's outer layer 43. A suitable material for the outer layer 43 is a fluid-impervious material, such as a polyethylene film. Each sanitary pad 10 is arranged such that its width 12 extends between a pair of outer margins 46.

The outer layer 43 extends outward in the transverse direction beyond the outer margins 46 on both sides of the sanitary pad 10. The process of making the strip 40 includes making two folds in the outer layer 43. Each such fold is along one of the two outer margins 46. This results in a longitudinally-extending overlap 55 in which the outer layer 43 overlaps itself. In some embodiments, application of heat or an application of an adhesive forms a longitudinal bond 56 that holds the overlap 55 in place. This longitudinal bond 56 can best be seen in FIG. 3A, which is a cross-section along the line B-B in FIG. 3.

A perforation 6 extending in the transverse direction penetrates all layers of the continuous strip 40. This makes it easy for a user to tear off the distalmost attached segment 5.

Referring to Section B-B in FIG. 3A, the manufacturing process results in layers 57 that form edge folds 54. These edge folds 54 create a barrier 58 against contamination and water ingress through the outside edge.

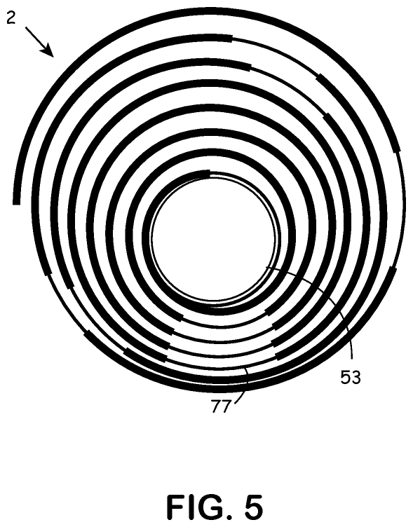

As shown in FIG. 2B, the seal 15 is much thinner than the absorbent region 14. This creates an irregularity in the sanitary pad's profile. This irregularity impedes the ability to roll the strip 40 to form the sanitary-pad roll 2. It does so, in part, because the seal's lack of rigidity makes it incapable of supporting what lies radially outward from it. As a result, if the seals 15 align along the same radial vector when rolled onto the sanitary-pad roll 2, something analogous to a sinkhole results. This causes an overall eccentricity in the sanitary-pad roll 2, as shown in FIG. 5.

The edge fold 54 shown in FIG. 3A adds enough height to function as a shim, as can be seen by comparing FIG. 2B and FIG. 3A. This results in a more uniform thickness that promotes the ability to form the sanitary-pad roll 2.

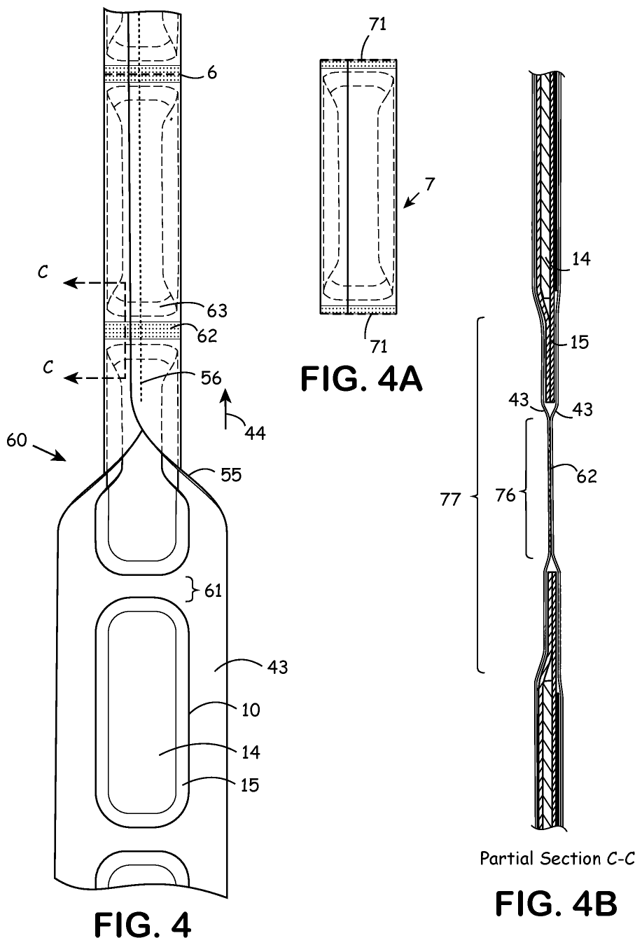

Referring to FIG. 4, the assembly of one version of a continuous strip 40 includes placing die-cut sanitary pads 10 on the outer layer 43 along the rolling direction 44 with each pair of sanitary pads 10 being separated by an inter-pad space 61.

At a folding region 60, the outer layer 43 is folded to form the overlap 55. In a preferred embodiment, the overlap 55 is heated or glued to form the longitudinal bond 56.

In some embodiments, it is useful to also form a transverse bond 62 that extends transversely across the inter-pad space 61. The transverse bond 62 is particularly useful to prevent a distalmost attached segment 5 from having an exposed transverse edge. The transverse bond 62 is made by either mechanically staking, heating, or gluing at the inter-pad space 61, thus causing the outer layer 43 to bond to itself. The transverse bond 62 forms a compartment 63 that protects the sanitary pad 10 that is sealed within it from dust or contaminants. The result is the attached segment 5 that was shown in FIG. 1.

The inter-pad space 61 is then weakened to promote the ability to separate the distalmost attached segment 5 from the sanitary-pad roll 2. This is typically carried out by perforating the layers of material to form a perforation 6.

Referring to FIG. 4A, after having been separated from the sanitary-pad roll 2, the detached segment 7 will have a torn or perforated edge 71 on each end. The detached segment 7 can then be unwrapped to obtain the sanitary pad 10.

FIG. 4B shows a cross-section C-C through that portion of FIG. 4 that includes the inter-pad space 61. As is apparent, there exists a discontinuity 77 in the thickness of the strip 40. In particular, the thickness of an absorbent region 14 far exceeds that of the inter-pad space 61. The discontinuity 77 also includes an embedded discontinuity 76 within it. This embedded discontinuity 76, which is typically only 0.04 millimeters thick, comprises only the outer layer 43.

FIG. 5 shows height discontinuities 77 that have aligned unfavorably when forming a sanitary-pad roll 2. This can occur when a radial vector intersects multiple discontinuities 77. As a result, the sanitary-pad roll 2 develops an eccentricity. This causes manufacturing difficulties whose severity depends on the difference between the thinnest and thickest sections of the strip 40 and the lengths of the discontinuities 77. To avoid these difficulties, it is preferably for the discontinuities 77 to be as short as possible and for the height differential caused by the discontinuity 77 to be as small as possible.

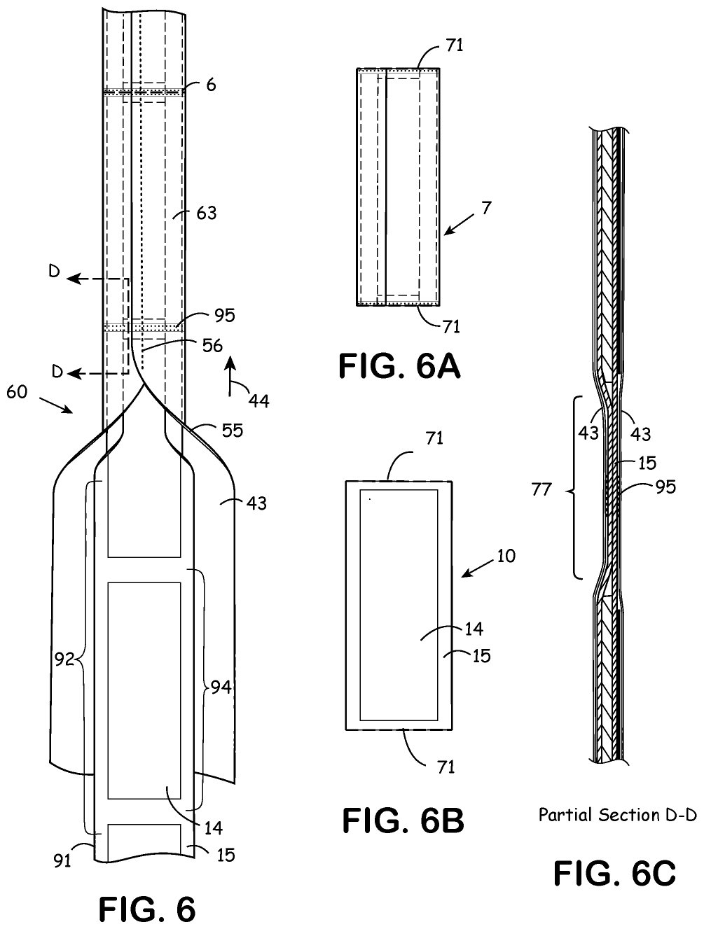

FIG. 6 shows an embodiment that avoids discontinuities 77 of the type shown in FIG. 4B. In the embodiment of FIG. 6, the strip 40 comprises a pad-bearing web 91 on which a series 92 of sanitary pads 10 has been placed along the rolling direction 44. The pad-bearing web 91 is placed on an outer layer 43 along the rolling direction 44. Each sanitary pad 10 has an absorbent region 14 surrounded by sealing region 15, as was the case in the embodiment shown in FIG. 4B. However, the pitch 94 with which the sanitary pads 10 are placed in this embodiment is equal to the length of a single segment 5.

At a folding region 60, the outer layer 43 is folded to form the overlap 55. In some embodiments, application of heat or an application of an adhesive forms a longitudinal bond 56 that holds the overlap 55 in place.

In some embodiments, it is useful to form a transverse bond 95. The transverse bond 95 is formed by mechanically staking, heating, or gluing at the inter-pad space 61 so that the outer layer 43 bonds to the seal 15 between each segment 5. This, in turn, creates a compartment 63 that protects the sanitary pad 10 from dust or contamination. Subsequently, the layers of material between each pad segment 5 are punched to form perforations 6 or otherwise weakened to allow easy separation of the distalmost attached segment 5 from the continuous strip 40.

Using conventional assembly machines, it is possible to cut a segment 5 from a roll of continuous absorbent material 23 and to time the bonding operation so that the transverse bond 62 is immediately between the materials of sealing regions 15 with a space between each segment 5. This is carried out in a rolling process without special machinery to handle the individual cut component. The foregoing method also avoids waste material if the segment 5 is rectangular. The foregoing method also avoids the delay associated with having to die cut a sanitary pad and thus permits more rapid manufacture.

In some embodiments, the transverse bond 62 is a thin strip. This geometry reduces stress on the seal 15 when a user of the sanitary pad 10 pulls the outer layer 43 away.

As shown in FIG. 6A, the detached segment 7 will have a torn or perforated edge 71 on each end after separation. This segment 7 can then be used like a standard sanitary pad by unwrapping the outer layer 43 and using the sanitary pad 10 within. The resulting sanitary pad 10, shown in FIG. 6B after having been removed from the detached segment 7, is a rectangular structure having an absorbent region 14 in the center that has been surrounded by a sealing region 15 and that has torn edges 71 on either end.

FIG. 6C shows a cross section along the line “C-C” in FIG. 6. As is apparent, the resulting discontinuity 77 is significantly smaller than that shown in FIG. 4B. This configuration is thus less likely to cause the eccentric sanitary-pad roll 2 shown in FIG. 5.

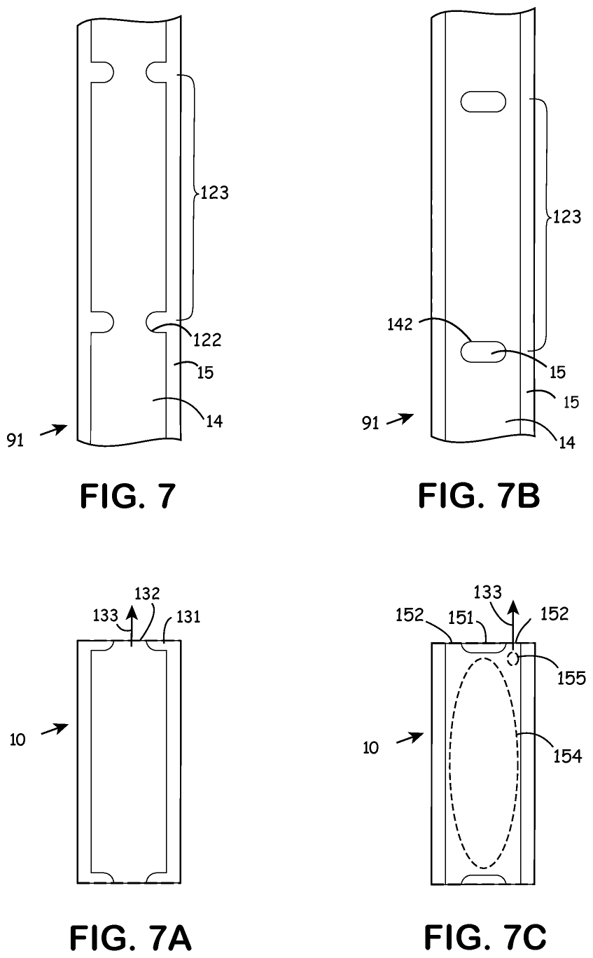

Referring to FIG. 7, an alternative pad-bearing web 91 features a continuous absorbent region 14 with notches 122 at regular intervals 123. These notches 122 mark boundaries between adjacent sanitary pads 10. In a rolling assembly process, a continuous absorbent region 14 as shown in FIG. 7 increases production speed.

Referring to FIG. 7A, the sanitary pad 10, after having been separated from the pad-bearing web 91, has a seal at each of its corners 131. However, an inter-corner space 132 between the corners 131 allows fluid 133 to leak.

In another embodiment, shown in FIG. 7B, center-punched holes 142 along the pad-bearing web 91 enable seals 15 to be formed at regular intervals 123 corresponding to the lengths of the sanitary pads 10.

As shown in FIG. 7C, a sanitary pad 10 from the pad-bearing web 91 shown in FIG. 7B comprises a sealed center section 151 and unsealed corners 152 through which fluid 133 that has been absorbed by the absorbent region 14 can leak out.

In the embodiment shown in FIG. 7C, as fluid 154 spreads out from the absorbent region 14, it eventually reaches corner regions 155 that are near the unsealed corners 152. These corner regions would be the furthest from the center of the absorbent region 14. Thus, if, as a compromise to promote ease of manufacturer, one had to choose a portion of the sanitary pad's edge to be unsealed, the corners 152 would be a better choice than the center section 132 shown in FIG. 7A because the corner regions 155 are furthest from that portion of the absorbent region 10 that would be expected to be the most swollen with absorbed fluids.

The details of an economical manufacturing method of these rolls such as materials, bonding, adhesives, methods for attachment to a roll, perforating, and die-cutting have been left out for clarity, and it is assumed that those skilled in the art of continuous fabric or paper manufacturing should be able to produce the rolls described.

It is to be understood that the foregoing description is intended to illustrate and not to limit the scope of the invention, which is defined by the scope of the appended claims. Other embodiments are within the scope of the following claims.

1. A manufacture comprising a sanitary-pad roll, said sanitary-pad roll comprising a core and a strip wrapped around a core, said strip extending along a longitudinal direction that is perpendicular to a transverse direction and comprising an impermeable layer, an absorbent layer, and a weakened region that extends along said transverse direction and defines attached segments that, when detached from said strip, form corresponding sanitary pads, wherein said attached segments include a distalmost attached segment, said distalmost attached segment being attached to only one other attached segment, wherein said impermeable layer, which is wider than said absorbent layer, is folded over along said longitudinal direction to form an overlap, wherein said impermeable layer comprises a backing material that is bonded to itself along said longitudinal direction to form said overlap, wherein said backing material and transverse bonds that extend across an inter-pad space between adjacent ones of said segments form individual compartments for each of said sanitary pads, each of said compartments being a sealed compartment that extends between said transverse bonds, whereby, as a result of being within said compartment, said segments, including said distalmost attached segment, are prevented from having an exposed transverse edge and said sanitary napkins in said compartments are protected from becoming wet and contaminated.

2. The manufacture of claim 1, wherein said weakened region comprises perforations that extend through said strip.

3. (canceled)

4. The manufacture of claim 1, wherein said sanitary pad is die cut.

5. The manufacture of claim 1, wherein said sanitary pad comprises wings.

6. The manufacture of claim 1, wherein said overlap is bonded.

7. The manufacture of claim 1, wherein said absorbent layer comprises a plurality of absorbent regions separated by inter-pad gaps, each of said absorbent regions corresponding to a sanitary pad, wherein said weakened region extends across said inter-pad gap.

8. (canceled)

9. The manufacture of claim 1, wherein said strip comprises a first segment comprising a first seal, a second segment comprising a second seal, and a length that extends between said first and second seals, wherein said length has a thickness that is less than that of either said first and second seal, and wherein said first and second seal has a thickness that is less than that of said absorbent layer.

10. (canceled)

11. The manufacture of claim 1, wherein each segment comprises a first and second end, wherein said second end is separated from said first end along said longitudinal direction, wherein said strip comprises a seal that extends from said first end to said second end, and wherein said weakened region extends across said seal.

12. The manufacture of claim 1, wherein said strip comprises a seal that separates adjacent segments and wherein said seal has a thickness that is less than that of said absorbent layer.

13. The manufacture of claim 1, wherein said strip has a length along said longitudinal direction, wherein said strip has a thickness that varies periodically along said length at a spatial frequency that is a function of the number of sanitary pads on said strip and said length.

14. The manufacture of claim 1, wherein said strip has a thickness that varies along said longitudinal direction, said thickness having a first value along a sanitary pad and a second value between sanitary pads.

15-20. (canceled)

21. The manufacture of claim 1, wherein said sanitary pad is not die cut and is formed by tearing across said strip at said weakened region.

22-24. (canceled)

25. The manufacture of claim 1, wherein said absorbent layer is discontinuous along said longitudinal direction of said strip.

26. (canceled)

27. (canceled)

28. The manufacture of claim 1, wherein each of said sanitary pads is surrounded by a seal that prevents leakage of fluids out of said absorbent region.

29. The manufacture of claim 1, wherein the number of transverse bonds is equal to one more than the number of segments.

30. The manufacture of claim 1, wherein said sanitary napkins lie entirely flat on their respective segments and wherein absorbent layers of said sanitary napkins are unfolded.

31. A method comprising making a roll of sanitary pads, wherein making said roll comprises making a strip of said sanitary pads, wherein said strip extends along a longitudinal direction that is perpendicular to a transverse direction, wherein said strip comprises an impermeable layer that comprises a backing material, an absorbent layer and a weakened region that extends along said transverse direction and defines attached segments, each of which contains a sanitary pad therein, wherein said impermeable layer is wider than said absorbent layer, wherein said attached segments include a distalmost attached segment, said distalmost attached segment being attached to only one other attached segment, wherein, between each pair of adjacent segments, there exists an inter-pad space across which said backing material extends, folding said impermeable layer along said longitudinal direction to form an overlap, bonding said backing material to itself, forming a sealed compartment around each of said segments, including said distalmost segment, wherein each of said sealed compartments protects a corresponding one of said sanitary pads from wetness and contamination, wherein forming said sealed compartments comprises, for each segment, forming a transverse bond across each of said inter-pad spaces adjacent to said segment, whereby each of said compartments extends between a corresponding pair of said transverse bonds, and wrapping said strip around a core.

32. The method of claim 31, wherein forming said transverse bond comprises mechanically staking at said inter-pad space.

33. The method of claim 31, wherein forming said transverse bond comprises heating at said inter-pad space.

34. The method of claim 31, wherein forming said transverse bond comprises gluing at said inter-pad space.
